(19) United States
(12) Patent Application Publication
(10) Pub. No.: US 2007/0038060 A1
(43) Pub. Date: Feb. 15, 2007

(54) Identifying and treating bodily tissues using electromagnetically induced, time-reversed, acoustic signals

(76) Inventors: Stephen A. Cerwin, Mico, TX (US); David B. Chang, Tustin, CA (US)

Correspondence Address: GENE W. ARANT, P.O. BOX 269, LINCOLN CITY, OR, 97367-0269, US

(21) Appl. No.: 11/450,097

(22) Filed: Jun. 9, 2006

Related U.S. Application Data
(60) Provisional application No. 60/689,216, filed on Jun. 9, 2005.

(51) Int. Cl. A61B 5/05

(52) U.S. Cl. 600/407

(57) ABSTRACT

A method of detecting and treating cancerous or cancer-supporting tissue by applying low-power density electromagnetic signals to the tissue, inducing acoustic signals from a portion of the tissue that exhibits an electrical conductivity gradient, receiving the thus-induced acoustic signals and from them identifying the location of the tissue portion, applying time-reversal to the thus received acoustic signals, accumulating the strength of the time-reversed acoustic signals, and then applying the thus strengthened acoustic signals to the identified tissue portion.

### PRIORITY CLAIM

This application claims priority of apparatus and methods disclosed in the following Provisional Application of Stephen A. Cerwin and David B. Chang, No. 60/689,216, filed Jun. 9, 2005, for “Method of Locating and Treating Cancer Using Electromagnetic Radiation, Induced Ultrasound Emissions, and Accumulated Time-Reversal of the Induced Ultrasound Emissions”.

### FIELD OF THE INVENTION

The field of this invention is medical apparatus and procedures for imaging, identifying, and treating bodily tissues.

### INCORPORATION BY REFERENCE

This application incorporates by reference essential information that is contained in Chang et al U.S. Pat. No. 6,535,625 issued Mar. 18, 2003; and Cerwin et al patent No. 6,974,415 issued Dec. 13, 2005, all in accordance with 37 C.F.R. 1.57(c).

### BACKGROUND OF THE INVENTION

It has long been known that cancer cells or lesions could be identified by their electrical conductivity. Later research indicates that within or bordering a cancerous tissue the electrical conductivity or impedance may vary (i.e., have a gradient). It is also known that acoustic power concentrated onto cancerous tissue will cause it to ablate or to be heated to destruction.

The Chang et al. and Cerwin et al. patents identified above describe a process referred to as “Electromagnetic Acoustic Imaging” (EMAI), in which electromagnetic signals are sent into bodily tissues under examination. Cancer cells or lesions along with some normal tissues are thereby activated to generate acoustic signals. These signals are then detected to identify the physical location of their sources. An important feature of the EMAI process is that the electric power densities of signals in the tissues during diagnostic procedures are within safe levels prescribed by federal agencies.

### SUMMARY OF THE INVENTION

The present invention provides apparatus and methods with which electromagnetic signals are sent into the bodily tissue under examination, and acoustic or ultrasound signals developed from tissues containing conductivity gradients. Those signals may then be processed and used to send energy back into the bodily tissue under examination, for purpose of medical treatment.

Further according to the invention, acoustic or ultrasound signals induced from tissue may be phase adjusted, reflected from a time-reversal mirror and then applied simultaneously with a radio pulse so that the two wave actions interfere, constructively or destructively, at the source of induced ultrasound. Experimental or medical treatment purposes can thus be served with EMAI offering visual evidence of treatment effectiveness.

Also according to the invention, the time-reversed acoustic signals may be repetitively amplified, obtaining increased power to apply to the offending tissue.

Still further according to the invention, the applied electromagnetic signals may be generated as a series of pulses. The augmented images of the acoustic or ultrasound signals may be synchronized with still oncoming radio signals to be applied to tissues.

As another feature of the invention, the applied electromagnetic signals may be continued for a substantial period of time with controlled frequency modulation, (i.e., “chirping”), which gives rise to still other ways of selectively distinguishing or combining the two different types of energy signals.

### DRAWING SUMMARY

FIG. 1 is a conceptual drawing illustrating some aspects of the apparatus and methods of the present invention.

FIG. 2 is a schematic outline of a system of apparatus that may be used in carrying out the present invention.

### THE CONCEPTUAL DRAWING

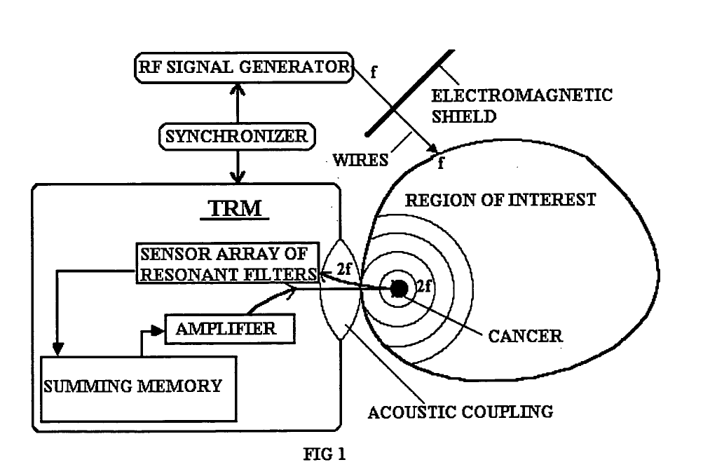

The conceptual drawing of FIG. 1 illustrates in a general way the apparatus necessary for carrying out the invention, and how it is intended to operate. The apparatus includes not only the essentials of an EMAI system as shown in the Chang et al. and Cerwin et al. patents, but also the essential parts of a time reversal mirror assembly.

This application incorporates by reference essential information from Fink U.S. Pat. No. 5,428,999 issued Jul. 4, 1995, and Berryman U.S. Pat. No. 6,755,083 issued Jun. 29, 2004, which describe the structure and operation of an ultrasonic time-reversal mirror assembly (TRM). According to the present invention the ultrasound signals induced from offending bodily tissues may be identified, amplified, and their directions reversed by a time-reversal mirror assembly before sending them back into some or all of the offending bodily tissues to provide medical treatment where needed.

Although not shown in the conceptual drawing itself, a complete system of apparatus needs to include a monitor for appropriate visual output of the information. Using the monitor a radiologist may select a region of interest which may include apparently abnormal or offending tissue, whether it be actual cancerous lesions, growths of veins or calcium deposits that surround cancerous tissue, blood vessels, or the like. In other words, a complete system of apparatus will provide the radiologist with ample opportunity to view possible targets in the region of interest and select one or more without involving adjacent tissue.

The system of apparatus includes a radio frequency signal generator which will generate radio waves at a known frequency f, within the range of about one-tenth to fifteen megahertz, which are then applied to bodily tissue through a known type of interface. In accordance with the EMAI process the electromagnetic energy received in the bodily tissue will induce ultrasound signals from tissues where conductivity gradients are found. The induced ultrasound signals will include a large component of energy at frequency 2f, double the frequency applied from the RF signal generator.

According to the invention the time-reversal mirror assembly, TRM, includes a sensor array of resonant filters for receiving the ultrasound signals and converting them into electronic form. In addition, the TRM also incorporates means for filtering and concentrating or amplifying those signals before reversing the direction of received ultrasound signals. The purpose of filtering is to more clearly identify the 2f energy component of the induced signals. The purpose of amplifying is to increase the energy level of the thus identified signals before applying them back to the bodily tissue for purpose of treatment. The filter and amplifier may be included as part of the TRM.

Auxiliary apparatus includes a synchronizer circuit to effectively control distributing, amplifying, and recombining pixel data generated by ultrasound signals received at the 2f frequency by the time-reversal mirror, as well as other associated signal processing requirements. Also included in the auxiliary equipment is an electromagnetic shield for protecting both the bodily tissue of a patient and sensitive instrumentation from uncontrolled or unwanted electrical signals.

### DETAILED DESCRIPTION

### DEFINITIONS FOR THIS PATENT APPLICATION

Ablate: “To remove by erosion, melting, evaporation, or vaporization.”
Coherence: “Maintenance of phase relationships between ultrasound transducer elements or sub-wavelength volumes of an ultrasound field from one event, such as receipt of an ultrasound signal between distinct time intervals after a fiducial time mark, to succeeding events following additional time marks.”
Depth of field of acoustic lens: “The distance between the nearest and farthest points of an object to a lens for which resolution in the image is of acceptable quality. For an ultrasonic lens this distance may be measured in microseconds of sound wave travel time between the two points.”
Depth of field of acoustic time-reversal mirror: “The distance or time required for resonant transducer elements to decay to acceptable relative levels after cessation of input ultrasound oscillations at a frequency near the center of the pass band of the elements.”
Lesion: “A localized pathological change in a bodily organ or tissue.”
Necrosis: “Death of cells or tissues through injury or disease, especially in a localized area of the body.”
Ray: “A thin directed line representing part of a wider radiation pattern.”

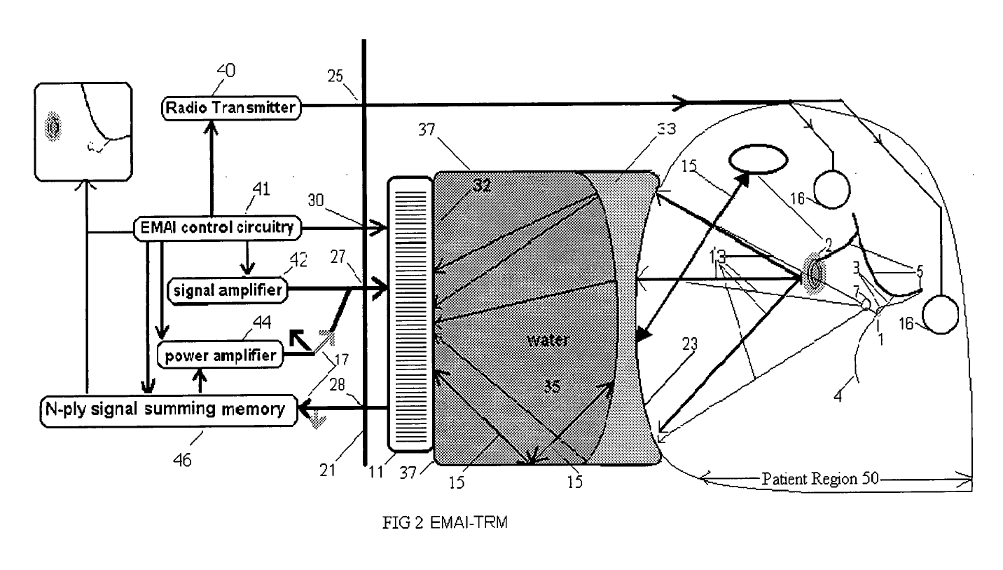

### PARTS LIST FOR FIG. 2

The various parts of apparatus and bodily tissue shown in FIG. 2 have the following part numbers:
1. Early stage cancer too small to see on this scale.
2. Large, later stage cancers with associated calcium deposits.
3. Tangled capillaries bringing blood to an early stage cancer.
4. Venule.
5. Arteriols.
6. Bone, scattering induced ultrasound.
11. Array of independent resonant ceramic (or other) ultrasound/electric (or optical) transducers. The usually accepted frequency range for ultrasound arrays or scanners is 2:1. The output may be presented on N separate channels or rapidly, sequentially scanned by line or frame, keeping the depth of field of the acoustic time-reversal mirror at a set limit consistent with or better than the depth of field of the acoustic lens.
13. Arrows indicating sound rays in the patients' body.
15. Sound rays that are reflected from the inside of the water container.
16. Electrodes on electrical coupling material in contact with the patient's body.
17. Ganged switches controlling the electrical output of the sensor array 11 through aperture 28 and the electrical input to array 11 through aperture 27.
19. Image display monitor.
21. Electromagnetic shield protecting the patient's body and array 11 from any uncontrolled electromagnetic radiation.
23. Acoustic lens interface of ultrasonic material matching to patient's body.
25. Aperture in shield 21 permitting controlled electromagnetic signals to enter patient's area 50.
27. Aperture sending reconstituted and amplified 2f electromagnetic signals to array11.
28. Aperture sending demodulated transduced 2f ultrasound signal amplitudes and phases to N-Ply signal summing memory 46.
30. Aperture in shield 21 permitting control signals to enter array 11.
31. Connection between monitor 11 and EMAI control circuitry that ensures simultaneous erasure of pixels in the monitor and corresponding locations in memory 46.
32. Window passing ultrasonic waves in either direction.
33. Acoustic lens having pressure-adjustable focal length.
35. Water volume allowing appropriate distance for focusing of ultrasonic waves.
37. Interface between water and air forming near-total reflection of ultrasonic waves in water.
40. Radio transmitter pulsed and frequency controlled by EMAI circuitry.
41. EMAI control circuitry that provides pulses of frequency ˜f to radio transmitter 40 and time delay phase adjustment through aperture 30 for carrier frequency 2f.
42. Amplifier of signals at 2f.
44. Power amplifier of signals at 2f.
46. N-Ply signal summing memory.
50. Patient region.

In drawing FIG. 2, the patient region is indicated on the right side, while most of the apparatus is on the left side of an electromagnetic shielding wall 21, needed to limit transmission of radio waves, protecting the patient as well as the ultrasonic sensor array 11. The array is composed of N independent transducers that convert ultrasound to electrical or optical signals and vice versa. It constitutes an operative portion of a time-reversal acoustic mirror assembly. Note that incoming signals directed to the inside of wall 37 would be near totally reflected with opposite phase. When array 11 re-emits the signal its phase would again be reversed on reflection by the wall so the wall causes zero total phase change.

A complete time-reversal mirror apparatus includes not only the sensory array 11, but also an “N-Ply signal-summing memory” 46 and “Power amplifier” 44. Numeral 23 on the forward or right-hand surface of array11 indicates a window, which, with aid of an acoustic impedance matching transmission powder or gel, passes ultrasound signals to or from the patient's body. Within the patient region, arrows indicate the paths and directions of signals. Array 11 can be a linear array or 2D array.

Radio transmitter 40 whose output, when it gets through shield 21 at aperture 25 and into the patient's body, is within safe power density limits required by federal regulations. A pair of electrodes 16, coated with conductivity enhancing ointment, represents a means for applying the low-power density electromagnetic signals to the patient's body. Alternatively, the RF energy can be fed into the body by means of currents in a coil; the oscillating magnetic field produces a localized electric field. Numeral 1, indicates an early stage cancer, virtually invisible on this scale, and 2 indicates volumes within the patient's body where a large lesion or cancerous tissue may exist. Numeral 3 indicates capillaries, which convey blood from arterioles, as at numeral 15 and to a plexus as near 1 where blood-tissue exchanges occur. Numeral 4 indicates a venule. All three kinds of vessels are critical to growth of aggressive cancers and all three respond to EMAI probe radio signals, announcing their presence through induced ultrasound emission at frequency 2f.

Aperture 28 in shield 21 permits a bundle of N or fewer electrical or optical signal filaments to pass from array 11 into the N-ply signal-summing memory 46 depending upon the scanning protocol adopted. If no scanning is used, then each of the N transducers drives a single filament and stores data in a timed sequence of pixels in memory 46. This is the nearly pure TRM mode with time modeling distance away from array 11. The time difference between pixels along the t axis determines the TRM range resolution limit. The time difference must be no smaller than the time required for the resonant transducer elements to ring down after a received ultrasound signal stops. It is a common practice for ultrasound transducer arrays to have a band-width approximately equal to its central frequency. If, for example, f=1 MHz then the central frequency, 2f, of the transducers means that the minimum resolution distance is ˜0.5 μs or 0.3 mm at a sound speed of 1500 m/s. If linear scanning is used, then for a square array only √(N) signal filaments would be needed and √(N) lines could be sequentially stored. This would make phase differences between lines and so distort the reversed emissions of the TRM. The control circuitry could remove this relative time delay in reverse emission, but the total time delay would increase the minimum resolved distance of the TRM by the scanning time, T times the number of scans: T×√(N). A ganged pair of up to N electrical/optical switches 17 control both the path of signals coming from array 11 into the memory and also signals leaving the power amplifier to be supplied to the transducer array 11 through aperture 27. The latter signals will cause array 11 to produce acoustic signals from its right side, radiating them back to converge high intensity ultrasound onto the tissue portions of the patient's body from which they arose.

An important feature of the invention is that the apparatus may be used for repetitively converging ultrasound onto sources selected according to their distance, t, from the sensor array and their x, y positions transverse to the t-axis. That the t-axis position is selectable to within t±DF/2, where DF is the depth of field of the lens and TRM systems, results from the near-transparency of many tissue organs to ultrasound. This causes the poor ultrasound reflection of such organs. The pressure-adjustable focus of lens 33 allows t-axis scanning.

Monitor 19 can show the input signal surface at the sensor array when time t after a short radio pulse is within ±half the depth of field for a given lens f# setting. By changing the pressure in a vacuum hose, not shown connected to the acoustic lens 33, the focus is adjusted nearer to or farther from conductivity gradients to image some normal tissue/cancers. After orienting herself or himself to the image data, comparing it with normal ultrasound images, the radiologist could decide on treatment. If ablation or cell necrosis by ultrasound heating is chosen she could then erase from monitor 19 images of tissues not to be targeted. If the “water” region 35 and acoustic lens 33 are present, then a 2D image of a conductivity gradient may be displayed on monitor 19. Reversible lines 31 connect an image pixel on monitor 19 to the corresponding memory elements in the “N-Ply summation memory”. As the erasing proceeds in monitor 19, this connection, if enabled, erases the corresponding memory elements at t±half the depth of field. The remaining “N-Ply signal summing” memory should then be directed by “EMAI control circuitry” to input the “Power amplifier”. Alternating the pressure in the vacuum hose scans the t-axis. In this way, all memory elements for tissue to be protected can be removed from focused emissions of the “Power amplifier”. Settings of N-Ply switches 17 will be changed to permit high power irradiation for treatment. After a large pulse is sent to the targeted conductivity gradients, switches 17 will automatically revert to their previous settings and further radio bursts will be provided to re-image the region of interest so the effects of treatment can be viewed.

In conjunction with the time-reversal accrual and amplification of acoustic signals, a critical requirement is that the correct phase relationship must be preserved; that is, the signals must be “coherent”. (See definitions.) This is facilitated by an overall phase adjustment sent by EMAI control circuitry through aperture 30. Digital handling of the signals in the “N-Ply signal summing” and “Power amplifier” are more accurate than earlier analog signal processing. The sequence of accumulations results in the following accumulated signal strength:

$$\begin{matrix}{S}_{Acc} = S [ 1 + G \dot{r} {ⅇ}^{- 2 aL} + {( G \dot{r} {ⅇ}^{- 2 aL} )}^{2} + {( G \dot{r} {ⅇ}^{- 2 aL} )}^{3} + … + {( G \dot{r} {ⅇ}^{- 2 aL} )}^{n - 1} ] \\ = {S [ 1 - {( G \dot{r} {ⅇ}^{- 2 aL} )}^{n} ] [ 1 - G \dot{r} {ⅇ}^{- 2 aL} ]}^{- 1}\end{matrix}$$

where G is amplifier gain, r is gain reinforcement at the conductivity gradient, a is the attenuation coefficient for ultrasound signal travel through unit length of the patient's body and L is the distance from a particular gradient to the sensor array 11. If the factor combination Gr·e^{-2aL} is close to unity as 1+epsilon where epsilon<<1 then

$${{S}_{ACC}}_{lim epsilon -> 0} = S [ n epsilon ] / [ epsilon ] = nS$$

For large n, this resonance condition can be quite large, growing by 10000/s, so less than a minute should be needed to accumulate enough power to ablate a tumor or melt a capillary closed.

As an alternative to [0027] above, “Radio Transmitter” 14 may repetitively induce acoustic signals from conductive spots within the region of interest. This signal data would be accumulated in the summing memory, allowing examination with the included monitor 19. An image of conductive gradients will appear and strengthen. As data from acoustic enhancements also accumulate, images of organ boundaries will gradually overlay the gradient data, aiding localization of gradients within organs. The radiologist should decide whether to use the repetitively accumulated data for ablative ultrasound heat treatment of conductive tissue. If the distance between transducer array 11 and the farthest point of the region of interest is 15 cm then it would take 97 microseconds to collect all signals from the entire region of interest for each radio burst. Allowing 100 microseconds for the period between repetitive electromagnetic pulses would mean that in one second 10,000 samples could be acquired. This would increase the signal to noise ratio, S/N, by a factor of 100 and the amplitude of the memory data by 80 dB. This assumes that “Signal amplifier” gain cancels acoustic attenuation between organ boundaries and array 11.

In applying the method of the present invention to early stage detection of breast cancer, very little acoustic reflection may be obtained from cancerous tissue, but acoustic waves are nevertheless obtainable by electromagnetic acoustic induction.

In accordance with the invention after acoustic signals identifying the location of a tissue portion have been received, the application of electromagnetic signals may be discontinued and the acoustic signals then amplified in a continuing loop path between the tissue portion and the time-reversal mirror.

### SENSING OBSTRUCTIONS IN BLOOD VESSELS

Obstructions may occur in such normal tissue as blood in capillaries or arterioles by abnormal tissues such as plaque accumulation, lesions or cancerous cells. Radiologists' experience should allow them to distinguish blood in normal vessels from abnormal tissue. Angiogenesis in breast tissue is taken as a sign that soon breast cancer may follow and the capillaries are targeted for ablation to prevent cancer growth. See FIG. 2, items 1 and 2, versus items 3 and 5. Items 3 show the start of early capillary entangling, an important cause of conductivity gradients near cancers and at other plexuses. The apparatus can be configured to co-register EMAI images with common ultrasound images—speeding radiologists' recognition of conductivity gradient tissues by their relation to organs and the telltale vessels feeding unseen, tiny but aggressive cancers.

One conductivity enhanced electrode is placed on the patient's skin over a vessel close to the surface as, for instance, at the ankle. The other is placed over a blood vessel plexus close to the skin such as the cardiac plexus. These electrodes will capacitively couple to the blood, completing an alternating current circuit. The electric field will be small in the high conductivity normal blood in veins or arteries, but will increase at any obstruction like plaque accrual in a vessel. The conductivity gradient there will induce ultrasound emission. Running a simple ultrasound-to-audio transducer over the leg, for instance, by noting the position of the most intense ultrasonic emission at the double frequency 2f, may sense this obstruction which might then be viewed by using the invented apparatus near the site. Care should be taken to avoid dislodging the obstruction as a whole. Sufficient power should be used to vaporize it to prevent formation of a dangerous obstruction in a narrower vessel as in heart muscle or the brain.

What is claimed is:

1. An apparatus for identifying and treating bodily tissues in the body of a medical patient, comprising:
(a) a signal generator for generating electromagnetic signals within the frequency range of about one-tenth to fifteen megahertz;
(b) means for applying those signals to a region of interest in the patient's body so as to induce ultrasonic signals at double the applied frequency;
(c) a time-reversal acoustic mirror assembly adapted to be positioned contiguous to the patient's body;
(d) an electromagnetic shield for limiting access of electromagnetic radiation to the acoustic mirror assembly;
(e) means to accommodate transmission of the thus-induced ultrasonic signals from the patient's body into the time-reversal mirror assembly;
(f) means for transmitting the induced ultrasound signals from the mirror assembly back into the region of interest of the patient's body; and
(g) control means operable for selecting a particular portion of the patient's body into which signals transmitted from the mirror assembly are to be sent.

2. Apparatus as in claim 1 wherein the means to accommodate the transmission of the thus-induced ultrasonic signals from the patient's body into the time- reversal mirror assembly includes an acoustic lens with a water region for focusing.

3. Apparatus as in claim 1 wherein the means for transmitting the induced ultrasonic signals from the time-reversal mirror assembly back into the region of interest of the patient's body includes an acoustic lens.

4. Apparatus as in claim 1 wherein the time-reversal mirror assembly includes means for summing results of repetitious occurrences of the induced ultrasonic signals.

5. Apparatus as in claim 4, which further includes means for amplifying the energy level of the thus-summed ultrasonic signals prior to their transmission back into the region of interest of the patient's body.

6. Apparatus as in claim 1, which further includes means for amplifying the induced ultrasound signals prior to their transmission back into the patient's body.

7. Apparatus as in claim 1, which further includes a visual readout apparatus associated with the control means for selecting a particular portion of the patient's body into which the ultrasonic signals are to be sent.

8. Apparatus for detecting and treating the bodily tissue of a subject, comprising:
(a) a radio frequency generator for generating signals at a known frequency within the range of about one-tenth to fifteen megahertz;
(b) means for applying the generated RF signals to the body of a subject;
(c) filter means for receiving from the body of the subject and selectively passing ultrasonic signals having about double the known frequency of the RF signals;
(d) time-reversal mirror mechanism cooperating with the filter means for reversing the ultrasonic signals passed by the filter means; and
(e) means for applying the thus-reversed ultrasonic signals back to the same bodily tissue of the subject.

9. Apparatus as in claim 8 which further includes means for amplifying the ultrasonic signals in addition to reversing them before they are applied back to the subject.

10. Apparatus as in claim 8 which further includes separate means for generating a reference signal at double the known frequency of the RF signal, for controlling the processing of the ultrasonic signals prior to their application back to the body of the subject.

11. Apparatus as in claim 10 which further includes means for amplifying the processed ultrasonic signals in addition to reversing them before they are applied back to the subject.

12. Apparatus as in claim 8:
(a) wherein the radio frequency generator is adapted to apply low-energy electromagnetic signals to the patient's body to induce acoustic signals from bodily tissue that exhibits an electrical conductivity gradient; and
(b) which further includes means for repetitively amplifying the ultrasonic signals passed by the filter means and moving them in a loop path between the bodily tissue and the time-reversal mirror mechanism.

13. A method of detecting and treating cancerous or cancer-supporting tissue by applying low-energy electromagnetic signals to the tissue, inducing acoustic signals from a portion of the tissue that exhibits an electrical conductivity gradient, receiving the thus-induced acoustic signals and from them identifying the location of the tissue portion, applying time-reversal to the thus received acoustic signals, accumulating the strength of the time-reversed acoustic signals, and then applying the thus strengthened acoustic signals to the identified tissue portion.

14. The method of claim 13 as applied to early stage detection of breast cancer, wherein very little acoustic reflection may be obtained from cancerous tissue, but acoustic waves are nevertheless obtainable by electromagnetic acoustic induction.

15. The method of claim 13 wherein after acoustic signals identifying the location of a tissue portion have been received, the application of electromagnetic signals is discontinued and the acoustic signals are amplified in a continuing loop between the tissue portion and the time-reversal mirror.

16. The method of claim 13 which is applied to locating blood vessel obstruction, comprising:
(a) applying an oscillating electric field at a frequency between 0.1 and 15 MHz to a portion of the blood vessel system of a patient's body;
(b) scanning a sub-portion of said blood vessel system by running an ultrasonic detector, operating at about double the frequency of said applied electric field, to a portion of said patient's skin treated to be ultrasound-transmissive; and
(c) noting the position of most intense ultrasonic emission at said double frequency.
